(19) United States
(12) Patent Application Publication
(10) Pub. No.: US 2023/0133253 A1
(43) Pub. Date: May 4, 2023

(54) INFLATABLE OCCLUDER APPARATUS AND METHOD FOR USING THE SAME

(71) Applicant: Motaz Shaher, Fort Wayne, IN (US)

(72) Inventor: Motaz Shaher, Fort Wayne, IN (US)

(21) Appl. No.: 17/514,137

(22) Filed: Oct. 29, 2021

(51) Int. Cl. A61B 17/12

(52) U.S. Cl. CPC A61B 17/12122; A61B 17/12136; A61B 2017/00871

(57) ABSTRACT

An assembly for in vivo repair of an atrial septal or like defect, including an occluder. The occluder further includes a hub, a waist connected in fluidic communication with the hub, a first lobe connected in fluidic communication with the waist, and a second lobe disposed opposite the first lobe and connected in fluidic communication with the waist. The occluder is non-metallic. The assembly further includes a catheter assembly, which includes a delivery tube having a proximal end and a distal end, a pressure sensor operationally connected to the delivery tube, and a hydrostatic fill material source connected in fluidic communication with the proximal end. The distal end is adapted to connect to the hub in fluidic communication for inflating/deflating the occluder.

### TECHNICAL FIELD

This novel technology relates generally to the field of medicine and, more particularly, to an occluder for repairing atrial septal defects, Patent Foramen Ovale (PFO), ventricular septal defects, patent ductus arteriosus, paravalvular leaks, vascular communications, or the like, that allows access to the chamber on the other side of the occluder.

### BACKGROUND

Catheter-based treatment for heart diseases is the fastest, and increasingly favorite, option for management of variety of common cardiac disorders such as atrial fibrillation, affecting about 2.3 million patients in the U.S., mitral valve repair for mitral regurgitation, affecting about 2% of the general population, and the like. These procedures are less invasive and better tolerated than alternative management options with overall better outcome. In the year 2020, over 38,000 left atrial appendage occluders were implanted in the US, over 31000 afib ablation procedures were performed, and an additional 80,000 percutaneous mitral valve clip procedures performed in the past few years. These numbers are expected to go up significantly in the next few years as doctors gain knowledge and experience in performing them.

What these cardiac problems have in common is that they affect the left atrium, a chamber that is difficult to reach with a catheter unless the doctor makes a hole in the atrial septum, the wall that separate the left from right atrium, resulting in iatrogenic Atrial septal defect (ASD). This hole enables the doctor to pass the catheter therethrough to the left atrium to perform the required procedure. Once the procedure is completed, over one third of these patients with iatrogenic ASD continue to have open ASD one year later. This number is also going up as larger holes are made to accommodate larger devices that are being implanted in the left atrium.

The residual ASD results in mixing of oxygenated and non-oxygenated blood, potentially causing low oxygen level, pulmonary hypertension, and eventually right sided heart failure. It can also be a conduit for a blood clot to travel to the brain from the venous side, resulting in stroke or worse. In addition, ASD has been associated with dangerous cardiac arrhythmia.

While surgery is a reasonable option in managing younger patients with congenital ASD, it is not appropriate in most patients with iatrogenic ASD due to the risk involved. So the remaining viable option most often contemplated is to use an ASD occluder device.

There are several ASD occluder known devices. These devices tend to be bulky and consist of nitinol (metallic memory) wire frame skeleton covered with a biocompatible membrane. These devices consist of a pair of relatively large self-expanding discs connected by a thinner waist. One disc is placed on the left atrial side and the other disc on the right atrial side of the hole while the waist spans the atrial septum. The discs are bulky and relatively stiff as they are meant to permanently block the ASD, forming a complete seal. However, as atrial septal defects are highly variable, the limitations of size and shape of the known occluder devices often means the matching of the device to the patient is less than ideal. As a result, in many cases there is at least some leakage. Moreover, it is very difficult to reposition the occluder device once the discs are deployed. While removal is possible, it is also very difficult due to the possibility of injury to adjacent cardiac structures from their metallic skeleton. Further, expanding stiff discs that can cause erosion to the atrial wall as well as potentially triggering arrhythmia due to irritation of the atrial wall. Because of the metallic skeleton, there is always a risk of perforation and pericardial effusion. Finally, there is a need for at least short-term anticoagulation treatment when implanting the known devices.

Despite potential risks of ASD and availability of these ASD occluder devices, their use is quite limited in patients with iatrogenic ASD and they are reserved to only patients with severe complications. A major contributor to the reluctance of doctors to use the known ASD occluder devices is the too common need to redo procedures, often requiring the need to make additional iatrogenic ASD. Currently, over half of the patients with afib ablation would go on to require a second ablation within one year. Likewise, most of the patients with mitral valve regurgitation will have recurrence of their disease within 7-10 years requiring another procedure. So, another puncture of the atrial septum and formation of another iatrogenic ASD is very common. This is why, the decision is made most of the time to leave an iatrogenic ASD open rather than risk an attempt to pass another catheter by making a hole in a large ASD occluder formed inside a metallic skeleton.

Because of this, doctors must be very selective in which patients have their iatrogenic ASD closed with an ASD occluder, and such procedures are only done sparingly in patients, typically those who are very high risk of low oxygen level and recurrent stroke. despite the potential risk involved.

This limitation does not only affect patients with iatrogenic ASD but also patients with congenital ASD/PFO. It is common to have afib in these patients in addition to mitral valve disease, which can present months or years after initial diagnosis of ASD. The decision to use one of the currently available PFO/ASD occluders often means that these patients will not able to undergo minimally invasive procedures that require atrial septal puncture because of the ASD occluders that have been implanted months or years before.

Thus, there is a need for an improved ASD/PFO occluder that may be easily repositioned or removed without excessive risk to the patient. The present novel technology addresses this need.

### BRIEF DESCRIPTION OF THE DRAWINGS

FIG. 1 is a first side elevation cutaway view of a first embodiment of the present novel technology, an inflatable hydrostatically supported atrial septal defect occluder device having a plurality of inflatable structural members disposed within an inflatable exterior membrane.

FIG. 2 is a front elevation cutaway view of the atrial septal defect occluder device of FIG. 1.

FIG. 3 is a front elevation view of a second embodiment atrial septal defect occluder device wherein the inflatable exterior membrane is the inflatable structural member.

FIG. 4 is a first side elevation cutaway view of a first embodiment of the present novel technology, an inflatable hydrostatically supported atrial septal defect occluder device having a plurality of inflatable structural members disposed within an inflatable exterior membrane wherein each portion of the device is independently inflatable.

FIG. 5 is a cutaway view of a section of the device showing multiple layers of different materials.

FIG. 6 is a partial cutaway view of the surface of the device showing a textured surface layer, multiple sublayers, and an inflatable chamber.

FIG. 7 is a cutaway view of a section of the device showing on-compliant layer embedded therein to define a predetermined shape un inflation.

FIG. 8 is a partial cutaway view of the surface of the device showing a compliant surface layer and a non-compliant sublayer that defines an inflated shape.

FIG. 9 is a front elevation cutaway view of the atrial septal defect occluder device of FIG. 1 showing multiple check valves operationally connected to each inflatable structural member and fused layers of different materials defining the inflatable exterior membrane.

FIG. 10 is a cross section of the waist of the device of FIG. 1 and showing multiple independent channels passing therethrough.

FIG. 11 is a front elevation cutaway view another embodiment of the atrial septal defect occluder device of FIG. 1 showing an inflatable structural ring operationally connected to and surrounding a side disc portion.

FIG. 12 is a cutaway top elevation view of the device of FIG. 1 showing inflatable structural members extending into both the left and right atrial portions and through the waist.

FIG. 13 is a first partial enlarged view of a structural member of FIG. 12.

FIG. 14 is a second partial enlarged view of a structural member of FIG. 12.

FIG. 15 is a side cutaway view of still another embodiment of the present novel technology, an atrial septal defect occluder device wherein each side portion includes a structural ring positioned thereabout.

FIG. 16 schematically illustrates the atrial septal defect occluder device of claim 1 as positioned blocking an atrial septal defect.

FIG. 17 schematically illustrates the atrial septal defect occluder device of claim 1 as positioned in a patient having PFO.

FIG. 18 schematically illustrates an occluder device embodiment having curved Tillable chambers within an outer membrane.

FIG. 19 schematically illustrates a hub portion of an occluder device embodiment having surface contours and magnetic portions for connection to a catheter.

FIG. 20 is a side cutaway view of an occluder device embodiment having contoured lobes connected by a bulged waist.

FIG. 21A-21C schematically illustrate an occluder embodiment having a fellable chamber portion for forming a skeleton.

FIG. 22 schematically illustrates a threaded hub portion of an occluder device embodiment having self-healing and two way valves for connection to a catheter.

FIG. 23 schematically illustrates a catheter operationally connected to the hub of FIG. 22.

FIG. 24 schematically illustrates a retrieval catheter operationally connected to the hub of FIG. 22.

FIGS. 25 and 26 schematically illustrate a catheter assembly for use with emplacing and filling an occluder device.

FIG. 27 schematically illustrates a retrieval catheter puncturing an occluder device.

FIG. 28 is a perspective vie of an occluder embodiment having an elongated waist.

FIG. 29A is a schematic view of a leaky valve.

FIG. 29B is a partial perspective view of a device lobe having curvature matching that of the leaky valve of FIG. 19A.

FIG. 29C is a partial side elevation view of the device of FIG. 19B.

FIG. 30 is a schematic view of an occluder embodiment having a single lobe.

FIG. 31A is a schematic view of a hubless occluder embodiment having a direct connection to a fluid source through a catheter.

FIG. 31B schematically illustrates the hubless occluder of FIG. 21A having ben filled and sealed.

FIGS. 32A-32F are perspective views of occluder devices having various shapes.

FIG. 33 is a perspective view of an occluder embodiment having a tapered waist.

FIG. 34 is a perspective view of an occluder embodiment having an elongated tubular shape.

FIG. 35 is a cutaway view of an occluder device having sequential fillable discs.

FIG. 36A is a cutaway view of an occluder device having multiple channels or tubules extending therethrough.

FIG. 36B is a cutaway sectional view of the waist portion of FIG. 26A.

FIGS. 37A-37H schematically illustrate the use of an occluder device having multiple fillable chambers for anchoring within a target organ.

FIG. 38A-38G schematically illustrate occluder embodiments having various waist shapes and contours for avoiding putting pressure on pressure sensitive tissues.

FIG. 39 schematically illustrates a shaggy occluder embodiment.

FIG. 40 schematically illustrates an occluder embodiment having a bulging disc portion and a shaped memory metal disc portion.

FIG. 41 schematically illustrates an occluder embodiment wherein the occluder is a plug.

FIG. 42 is a top elevation view of an occluder embodiment having a spiral shape.

### DETAILED DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

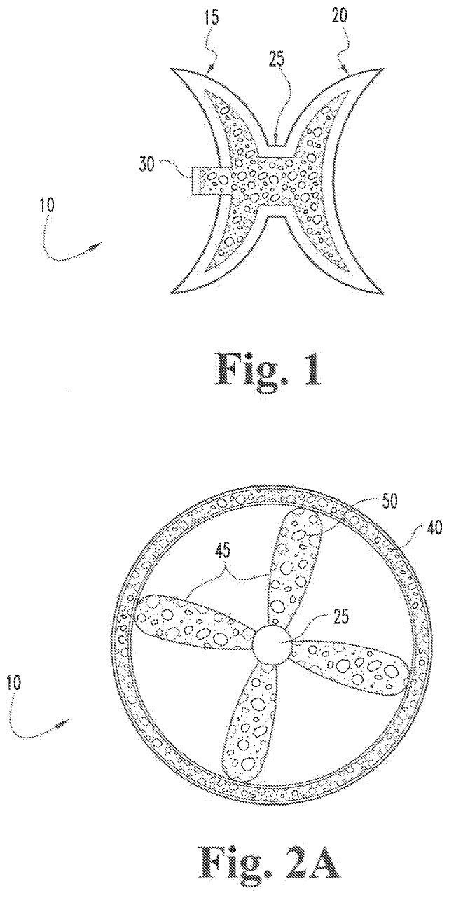

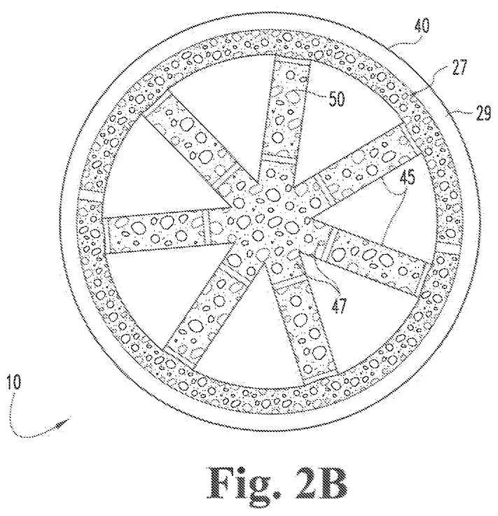

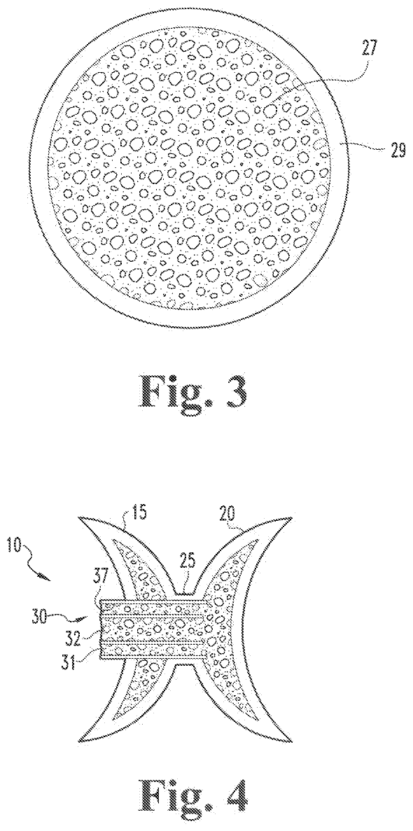

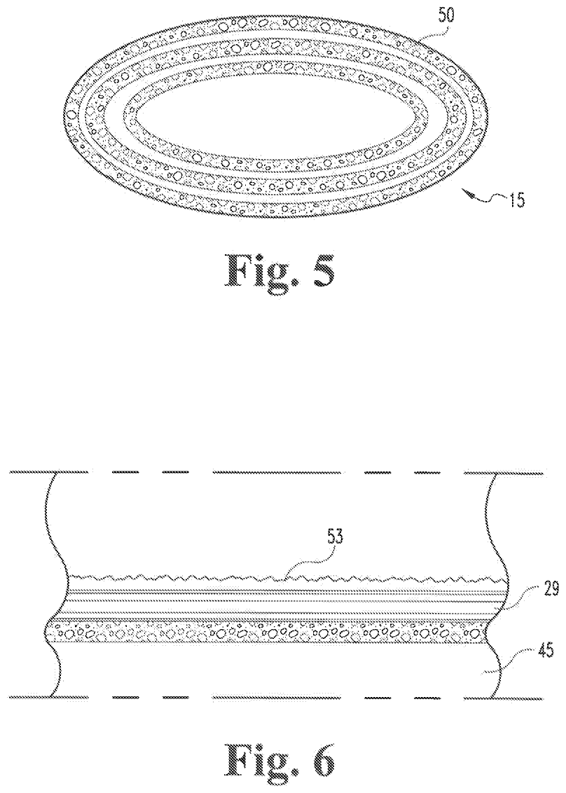

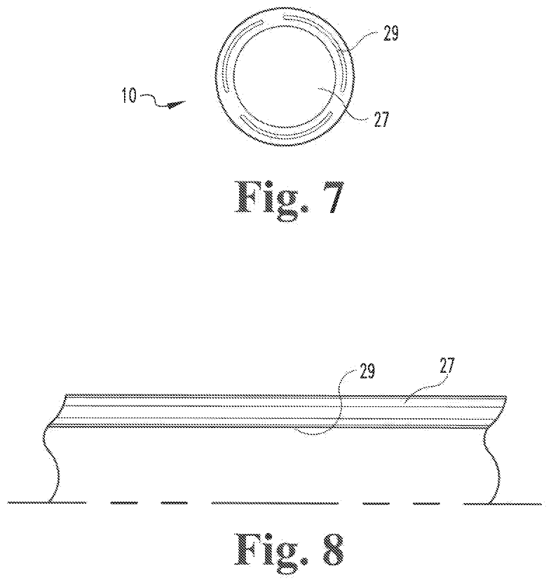

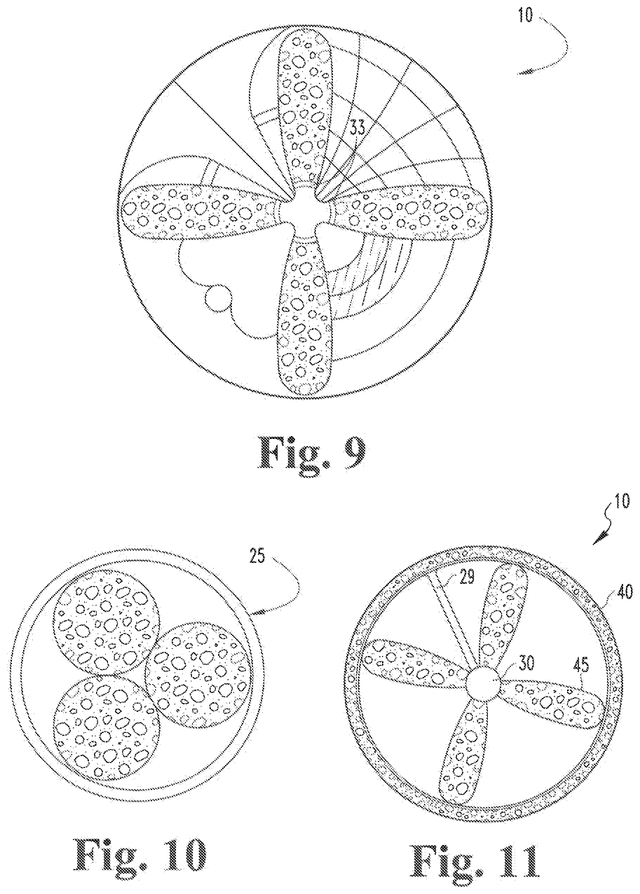

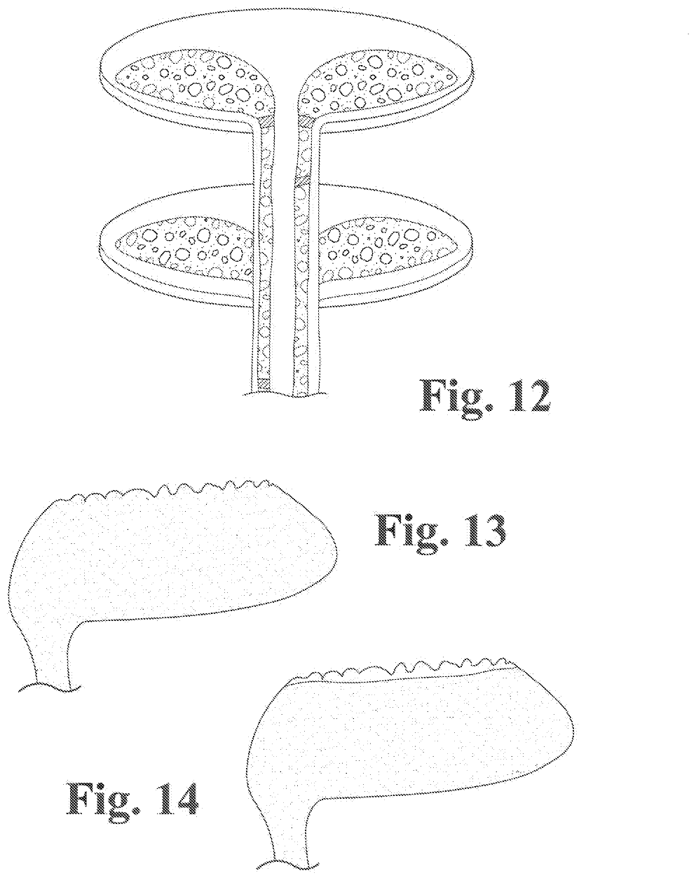

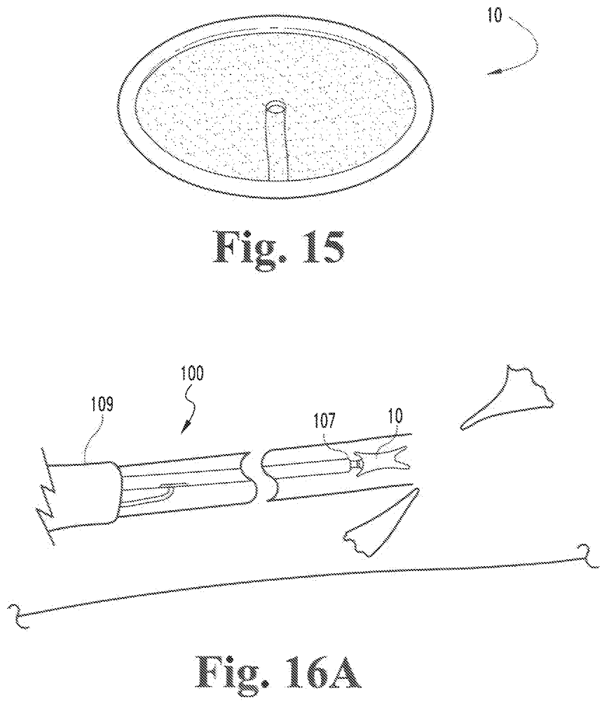

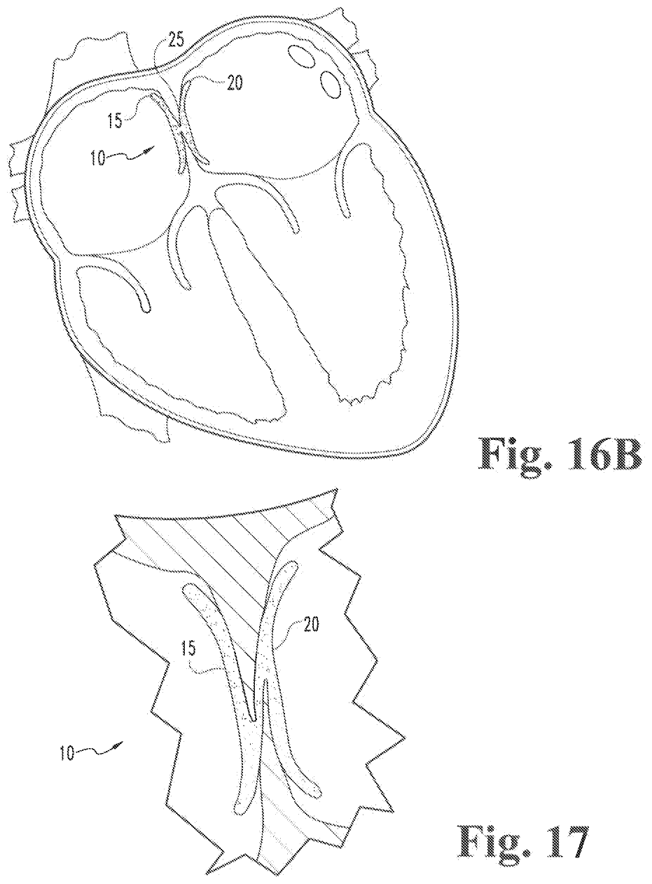

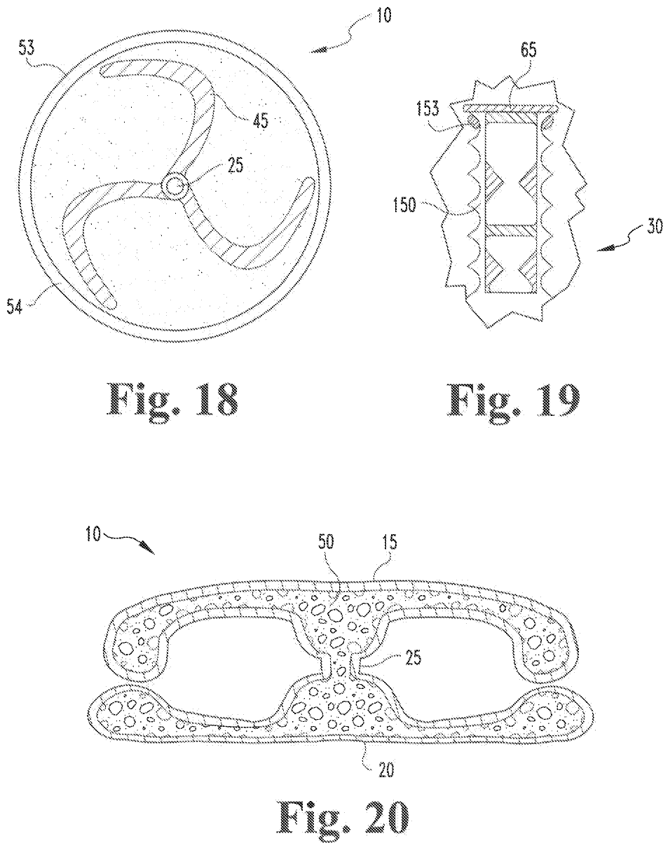

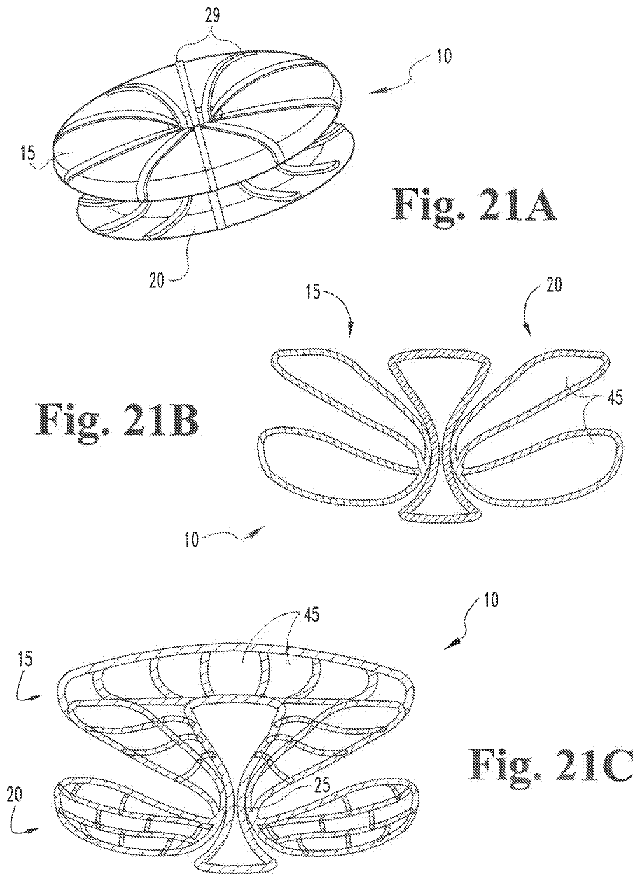

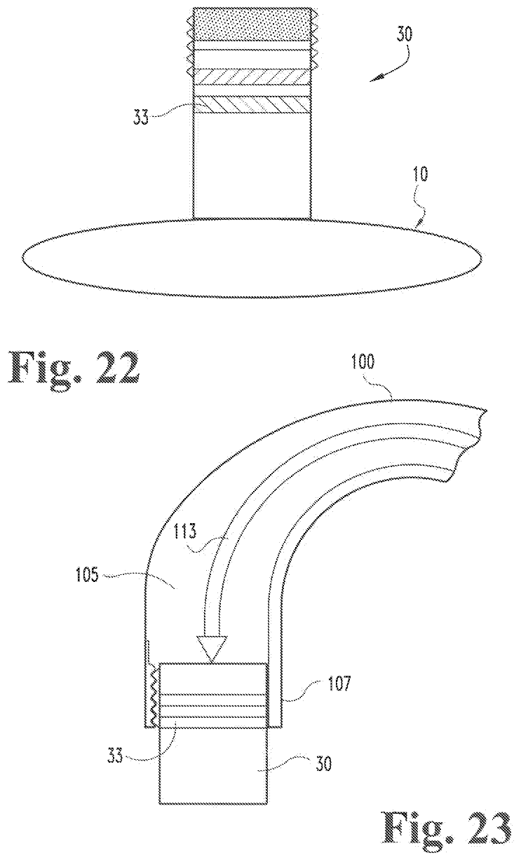

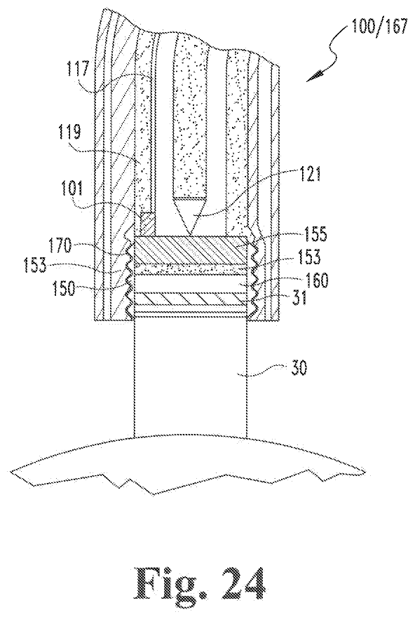

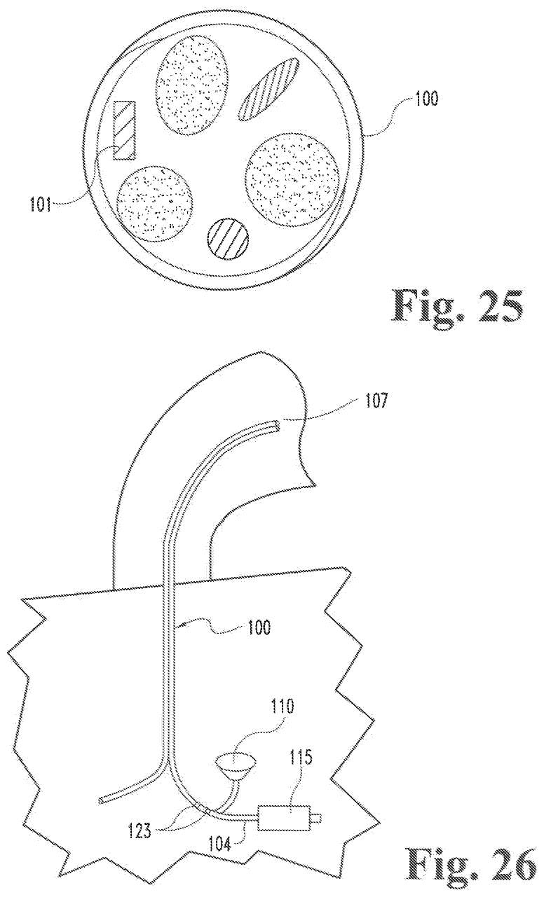

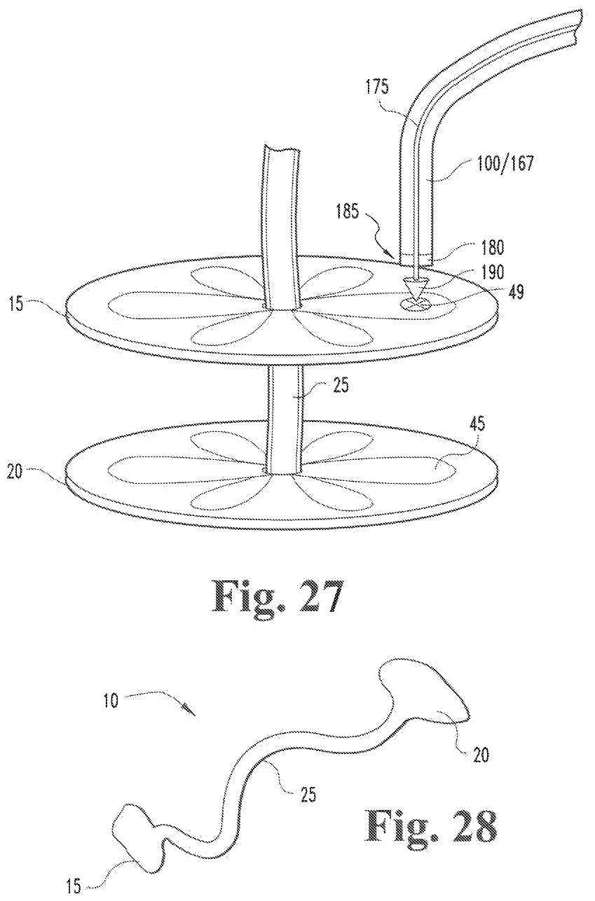

FIGS. 1-27 illustrate various embodiments of the present novel technology, an inflatable hydrostatically supported atrial septal defect (ASD) occluder device 10 including a left atrial side portion or lobe 15, a right atrial side portion or lobe 20, and a waist or connection portion 25 extending therebetween. Although left and right side lobes 15, 20 typically have the shape of discs, the discs are not necessarily equal in size and shape and one or both may be asymmetric. The occluder device 10 is not limited to the repair of ASDs, but may also be applicable to the repair of other aperture defects, such as patent foramen ovale, ventricular sepal defects, vascular openings, patent ductus arteriosus, and the like. The device 10 further includes at least one inlet hub or port 30 for introducing/removing fluid to inflating/deflating one more portions 15, 20, 25 of the device 10; the port 30 is typically positioned at the waist 25, but may be positioned at any convenient location. The hub 30 may include break-away connection, detachable valve and/or a quick release mechanism. The hub 30 may also include a self-closing valve, duck valve, flap valve or other medical valves. Hub 30 may be operationally connected to multiple occluders 10 which may be oriented inn parallel or sequential to each other. The hub 30 may feature two way valves to enable inflating and deflating the device(s) 10. The surface of the hub 30 may have additional padding or other materials for better attachment of a filling catheter and/or to enable better endothelial formation.

The hub 30 may have an internal chemical coating 37 that can fuse with opposing walls chemically or mechanically once inflation of the device 10 is complete. In some embodiments, the defect is sealed when one or both lobes 15, 20 are inflated against the surrounding tissue walls; in other embodiments, the defect is sealed when the waist 25 inflates sufficiently to fill the defect.

In some embodiments, the port 30 includes separate access channels 31, 32, each respective access channel 31, 32 in fluidic communication a different respective portion 15, 20, 25 of the device 10. Each portion 15, 20, 25, may include hydrostatically inflatable portions 27 and non-inflatable portions 29.

The device 10 further includes an outer portion 40 supported by inner support members 45. The outer portion 40 extends over atrial side portions and the hub 15, 20, 25 and defines the exterior of the device 10 that is in contact with the atria tissue and blood flow. The outer portion may be a single layer, multiple layers of the same or different materials and/or as an inflatable exterior bubble defining an interior volume. The inner support members 45 are positioned within the outer portion 40 and may be filled with hydrostatic support material 5o to give the inner support members 45 their shape and structural properties.

The outer surface portion 40 and the inner support members 45 may not have homogeneous compliance so as to predetermine and control the shape of the individua members 45 and overall device 10 when inflated with hydrostatic fill material 50. This may be accomplished by variations in materials, variations in thickness, variations in adhesion between layers, and/or the like. In some embodiments, the support members 45 and/or exterior portion 40 include multiple layers of different compliance properties to predetermine the inflated shape and size of the device 10.

Support members 45 and outer layer 40 (when inflatable) are connected to the valve 33 via channels 47, which may be unitary with the support members 45 or may be separate conduits for guiding and directing fill material 50. Such hydrostatic support material 50 may include liquids, semi-liquids, hydrogels, gases, gas bubbles, beads, foams, fluid polymeric material, saline solution, blood, liquid polymer, polyethylene glycols, polyphosphazene, polyacrylates, polydiacrylates, polyurethane, polyacrylamide, polyvinylpyrrolidone collage, carbohydrate, polylactic acid and the like and combinations thereof. All hydrostatic fill materials 50 are biocompatible, as leaks may occur. The hydrostatic fill material may be made radiopaque (such as with the addition of an iodine-based contrast material or the like) to enhance x-ray imaging, filled with micro-bubbles to enhance ultrasound viewing, or the like. Properties of the injectable material can be changed by injecting additional material that will change the pH, cause precipitation, solidification, coagulation, ionization, change mechanical properties of the initial injectable material, change properties of the initially injectable material change properties when exposed to light, heat, cooling, laser, pressure, blood, and the like.

The members 45 and channels 47 may be shaped and oriented to give shape and support to the disks 15, 20 and the waist 25. Typically, spaces are left between members and channels 45, 47 to accommodate puncturing if desired for moving and/or removing the device 10. The exterior surface may include a marker, such as a target 49, to guide a puncture tool to the desired puncture location. The device 10 is typically made of a pliable material to accommodate one or more punctures to accommodate repeat access if necessary.

The fluidic inlet valve 33 is positioned at the hub 30 and is typically a check valve to allow for unrestricted fluidic inlet (hydrostatic material 50 into the support members 45) but not allow egress of such materials 50 unless the valve is held open, such as by a filing needle or catheter. The inlet valve 30 may be any convenient check valve, such as spring loaded, magnetic, pressure sealed, or the like.

In some embodiments, the inlet port or hub 30 includes multiple fluidic inlets or pathways 31, 32, each respective channel connected 31, 32, in fluidic communication with a respective side 15, 20, waist 25, and/or member 45. Each respective inlet channel 31, 32 may be separately accessed to inflate or deflate a side or member 15, 20, 25, 45, independently of the others 15, 20, 25, 45, and may include one or more check valves 33 connected in fluidic cooperation. Channels 31, 32 may be provided as single conduits or as pluralities of conduits cooperating with one another. The device 10 may be valved to inflate the various chambers/channels 15, 20, 25, 31, 32, 45 sequentially, simultaneously, or in any predetermined order.

The device 10 is non-metallic, with the outer surface 40 and interior support members 45 made of, typically biocompatible polymer materials such as PTFE, compliant, semi-compliant or noncompliant materials such as latex, rubber, silicone, polyurethane, Polyethylene terephthalate, polyamide, Polyethylene terephthalate, polypropylene, fluroelastomer, plastic, or any elastic or inelastic material. The outer surface 40 is typically made of a softer, more compliant material that will conform to the surface of the atrium, while the inner members 45 may be made of a stiffer material that will better withstand the pressure of the fill material 50. The outer surface 40 may be smooth, contoured, roughened, and/or may include elongated structures extending therefrom to facilitate connection to the surrounding tissue. The device 10, especially the outer surface 40, may be made of biodegradable materials that dissolve over time to facilitate degradation over time and/or integration with surrounding tissue.

In some embodiments, small amounts of metal may be added to yield desired properties, such as a magnetic valve 30, a magnetic engagement of a delivery catheter, structural reinforcement, and the like.

The surface 53 is typically a membrane and may include an inflatable edge or perimeter 54, including one or more channels 47 for delivering and/or distributing hydrostatic support material 50.

The wall of inflatable chambers can be made from compliant material such as Polycaprolactone (PCI), Pollactic acid (PLA), Polydioxanone (PDO or PDS), Polyglycolic acid (PGA). Inflatable members/chambers/channels 15, 20, 25, 31, 32, 45, may be formed to take any predetermined shape, such as an H, X, Z, coil, or any like shape.

In some embodiments, the surface 53 of the device 10 may include one or more layers of medicinal coating 55, such as hiruidin, fibronectin, anticoagulant, antithrombotic, antimitogens, antimitotoxins, gene therapy, nitric oxide, hirulog, heparin or the like, and/or the coating 55 may include a biocompatible adhesive. In some embodiments, the surface 53 is roughened to facilitate adherence to the surrounding tissue. In other embodiments, the surface 53 contains filaments or tentacles 60 extending therefrom to better facilitate attachment to the adjacent tissue and/or to better seal the atrial defect.

In operation, the occluder device 10 is loaded into a delivery catheter 100. The device 10 is small enough to fit within delivery catheter 100. The catheter 100 may include a suction mechanism so as to facilitate attachment to the hub 30; likewise, the catheter and hub 30 may be matably threaded and/or magnetically coupled to facilitate connection. The catheter 100 is guided to the site of the atrial septal defect, such as by using a magnetic stereotaxis approach, and the device 10 is deployed, positioned, and inflated with hydrostatic fill material 50, such that respective sides 15, 20 are positioned on respective sides of the atrial septal defect with the waist 25 extending therebetween.

Catheter 100 includes a delivery tube portion 105 defining a cavity sufficiently large to enclose device 10 for in situ delivery of the device 10 through a distal end 107. Catheter 100 further includes a proximal end 109 for connection in fluidic communication with a hydrostatic fill material source 110. Catheter 100 may be an elongated straight member, may be curved or twisted, or may be of inconstant shape. In some embodiments, the delivery catheter 100 includes a first channel 105 for delivering the device 10 and a second channel 113 which may be used to transfer hydrostatic fill material no and/or to pump fluid therethrough. Pressure sensor 101 is disposed at or near the distal end 107, while a pressure monitor 115 operationally connected to the pressure sensor 101, such as via a wire 117, is disposed at or near the proximal end 109. Wire 117 may enjoy its own channel 119. First channel 105 may have a syringe 121 disposed at its proximal end 109, with one or more valves 123 connected in fluidic communication between the proximal and distal ends 109, 107.

The catheter 100 is operationally connected in fluidic communication with one or more valves 30, and the device 10 may be filled through one or multiple channels 31, 32, with all members 45 filled simultaneously or separately. The device 10 may simply be filled with hydrostatic fil material 50 until the respective side 15, 20, waist 25, and/or structural members 45 attain their predetermined inflated shapes and/or structural support characteristics, or, more typically, the respective side 15, 20, waist 25, and/or members 45 are inflated to respective predetermined pressures equating to the desired structural shape and support strength. The pressure within the respective side 15, 20, waist 25, and/or members 45 is monitored through the catheter 100, which includes a pressure sensor 101 operationally connected thereto. Target pressure may also be estimated based on the known properties of the respective side 15, 20, waist 25, and/or member 45 and the volume of material 50 injected. Inflation pressures may range from 0.00001 atmospheres to 1000 atmospheres, more typically from 0.01 to 1 atmospheres. The pressure used is based on the structural properties of the device, adjacent chamber pressure, adjacent tissue tolerance, and the like.

As more material injected into the device under pressure from the guide catheter through the hub, the volume and pressure go up in the connected chamber and/or channel, resulting in an increase in structural rigidity and formation of the desired shape. Shape is the result of radial and or longitudinal expansion based on the presence of compliant and noncompliant components of the inflatable chambers walls.

The device 10 may have to be partially deflated, repositioned, and re-inflated one or more times; the delivery catheter 100, if previously disengaged, is reengaged with the device 10 and hydrostatic fill material 50 is removed from the device 10 through the catheter 100 to deflate the device 10 to a predetermined size/pressure until the device 10 is sufficiently small to remove and/or reposition. Pressure within the members 45 may be measured through the catheter 100 connected in fluidic communication therewith. In some embodiments, the hub valve(s) 30 is/are self-sealing check valves. In some embodiments, a plug 65 is engaged to seal the hub/valves 30. In other embodiments, the hub valve(s) 30 may be sealed via a knot or clip, and in other embodiments the hub 30 is sealed via application of heat and/or cement and/or an adhesive. Once the device 10 is filled and properly positioned, the catheter 100 is disengaged from the hub 30 and withdrawn.

Another method to reenter the atrial septum in patients where the device 10 is stuck to the wall and cannot be safely removed, is by puncturing the device 10 in between the Tillable chambers 45 inside the left and right arial discs 15, 20 and making a new ASD through the space in between them. Likewise, the device 10 may be punctured using shape needle/device inside the fillable chambers 45 inside the left and right areal discs 15, 20 and making a new ASD through them. The device 10 may keep its shape because every fillable chamber 45 has its own valves 33 that prevent leaking from adjacent chambers 45. Moreover, if the device 10 defines a single unitary fillable chamber, the device 10 may be punctured and drained of hydrostatic material 50 and a new ASD may be formed through the deflated device 10. The device 10 will stay in place, and once the procedure is complete then additional ASD occluder 10 can be placed through the old one 10.

ASD device puncture can be done using fluoroscopy guidance or ultrasound (TEE and ICE) guidance or using fusion of different imaging modalities such as TEE and fluoroscopy, 3D echocardiography, CT derived 3D augmented fluoroscopy, real time MRI, or other imaging modality guidance. Iodinated contrast present in fillable chambers may help guide the puncture location.

Tools used to puncture through the ASD occluder 10 or the atrial septum include but are not limited to stainless steel needles, BRK needles, or the like, and may also include a needle-wire system, guidewire, Confida wire, Safari wire or other shape needles, wires or other sharp objects. Alternately, the puncture tool may use Radio Frequency (RF), NRG RF transseptal needle, or other needles using RF, laser, heat or other forms energy to achieve puncture.

After performing puncture then a guide wire may be advanced through the device then a sheath can be advanced into the left atrium using the guide wire. If larger sheath needs to be used, then further dilation of this defect can be done using dilator or balloon septostomy.

After completion of the procedure such as afib ablation or mitral valve repair then another ASD occluder can be implanted across the preexisting ASD occluder in a fashion similar to original technique

Inflatable elements can define a skeletal matrix of tubes, channels or fillable chambers 31, 32 in any particular pattern, radially, circular, curvature. They can in between the connecting tubes. This will enable the device 10 to unfold appropriately. These inflatable elements 31, 32 can be encased inside the device 10. Once these inflatable elements 3, 32 are inflated at pressure and volume as described above then the device 10 gets its shape. Typically, fillable chambers 45 in the left and right atrial discs 15, 20 are oriented to be disposed parallel to each other 45 so that if the device 10 needs to be punctured, a puncture may be made through a space between the fillable chambers 45 in the right atrial disc 20 and continue to pass through a similar space in the left atrial disc 15.

In some embodiments, the hub 30 is externally threaded 150 and/or equipped with a magnet/magnetizable portion 153, and includes one or all of the following: a seal or plug 65 at its terminal end 155, a self-healing valve 160, a 2-way valve, and/or a check valve 33. Retrieval catheter 167 may be internally threaded 170, with internal threading matable with hub external threading 150 and/or include a magnetic/magnetizable portion 153 matable with the hub magnetic portion 153. Retrieval catheter 167 further includes a suction line 175 terminating in a suction port 180 disposed at the distal end 185 of the catheter 167. Catheter 167 further includes a puncture tool 190 disposed at or near the distal end 185. Puncture tool 190 may be a sharpened elongated member, an RF delivery guide, or the like. Once engaged with the hub 30, retrieval catheter 167 may be operated to puncture the hub seal 65 with the puncture tool 190 and deflate the device 10 by removal of hydrostatic material 50 through the suction port 180 and suction line 175.

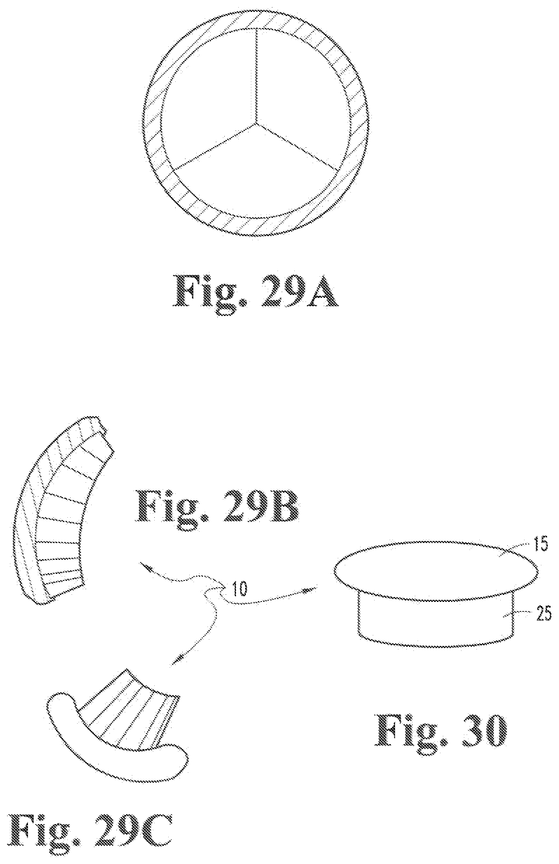

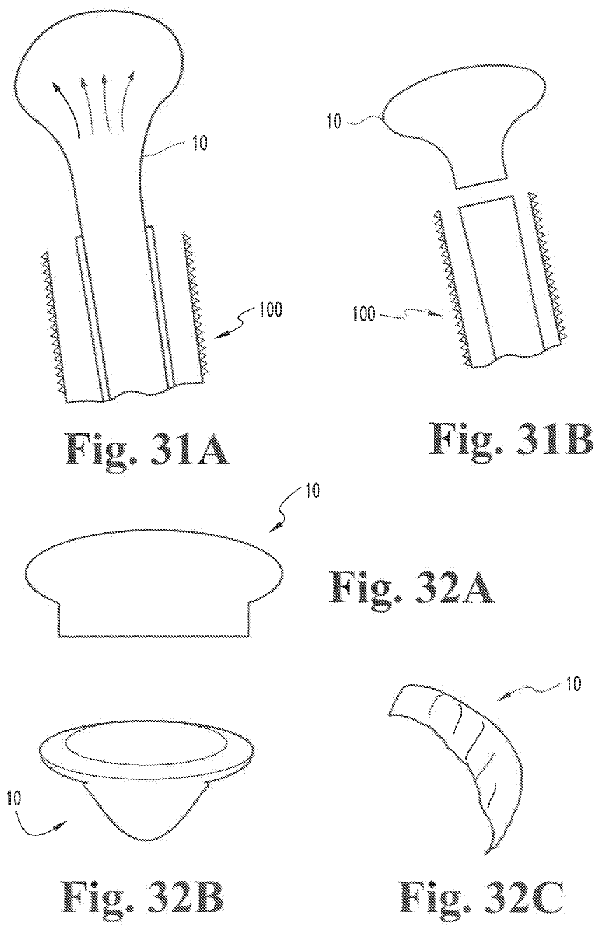

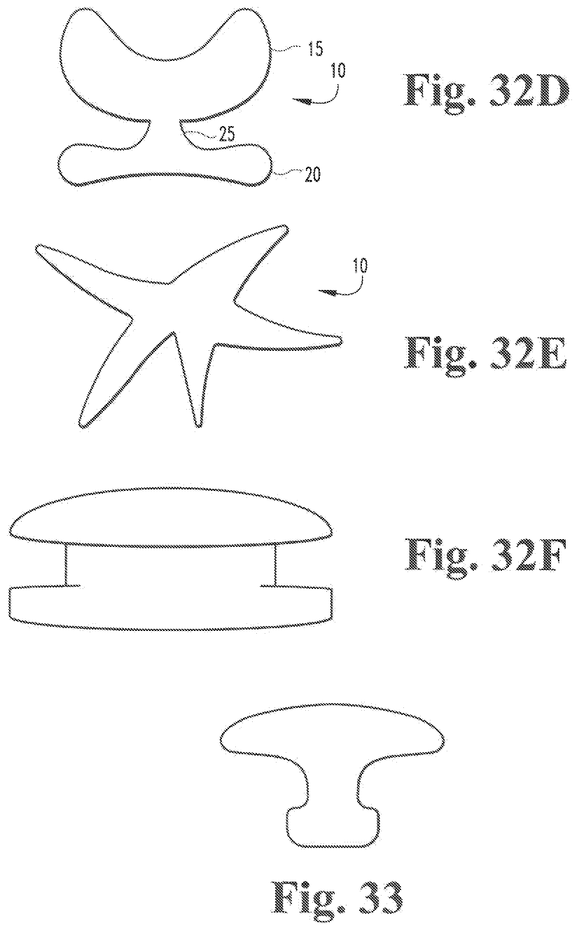

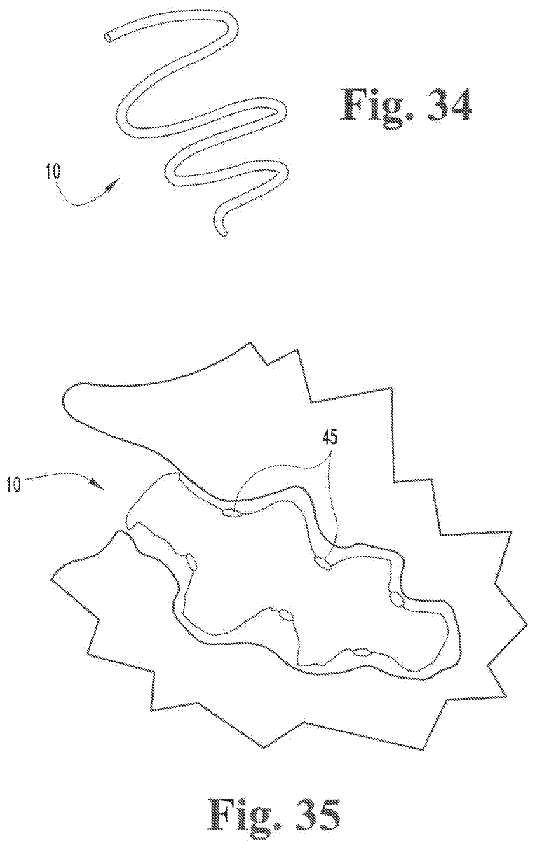

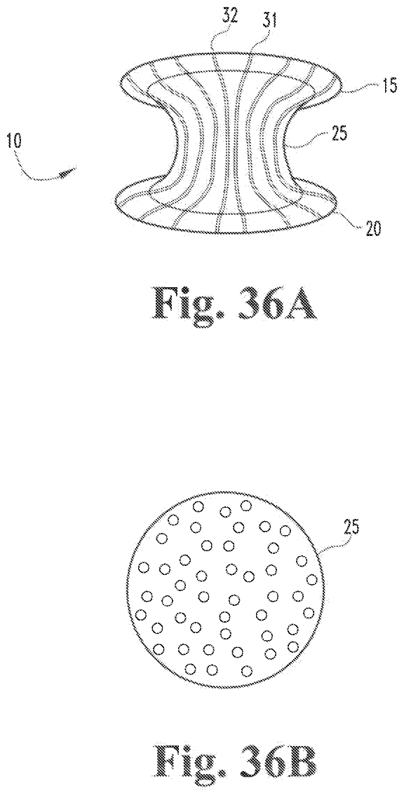

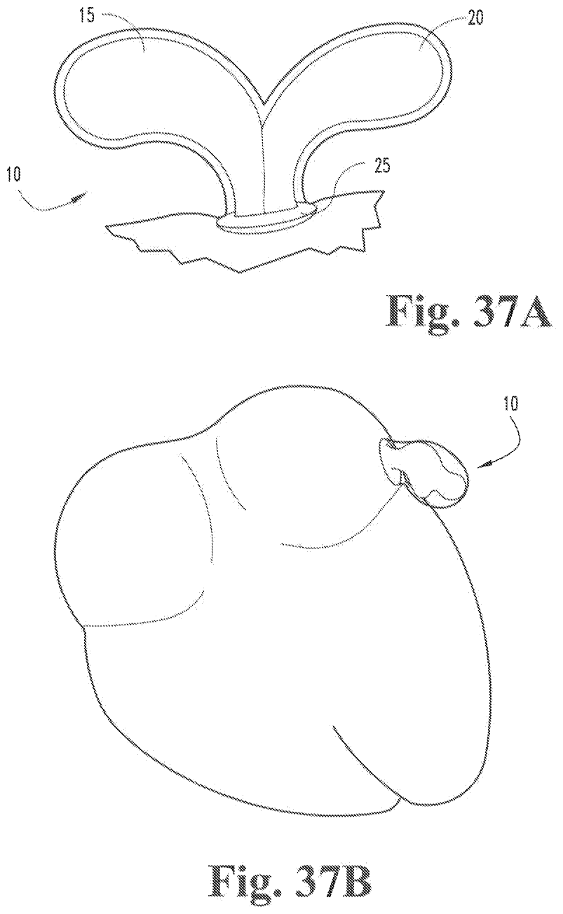

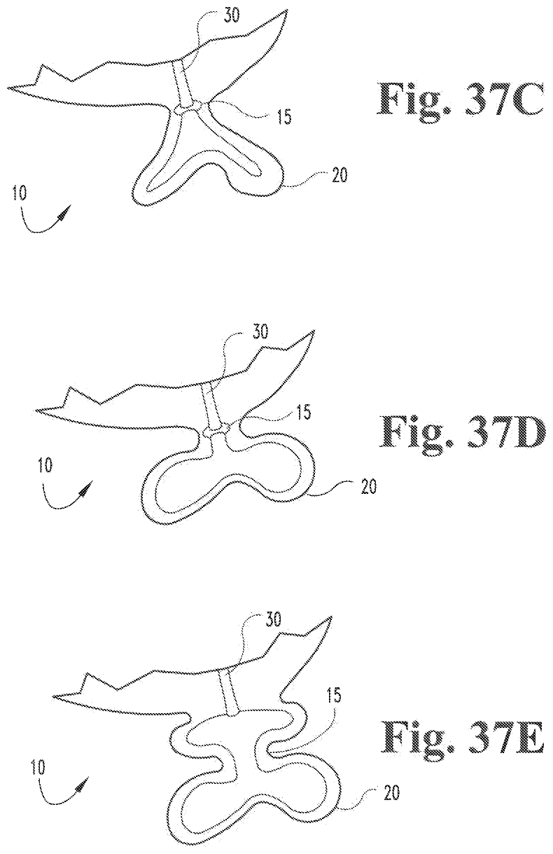

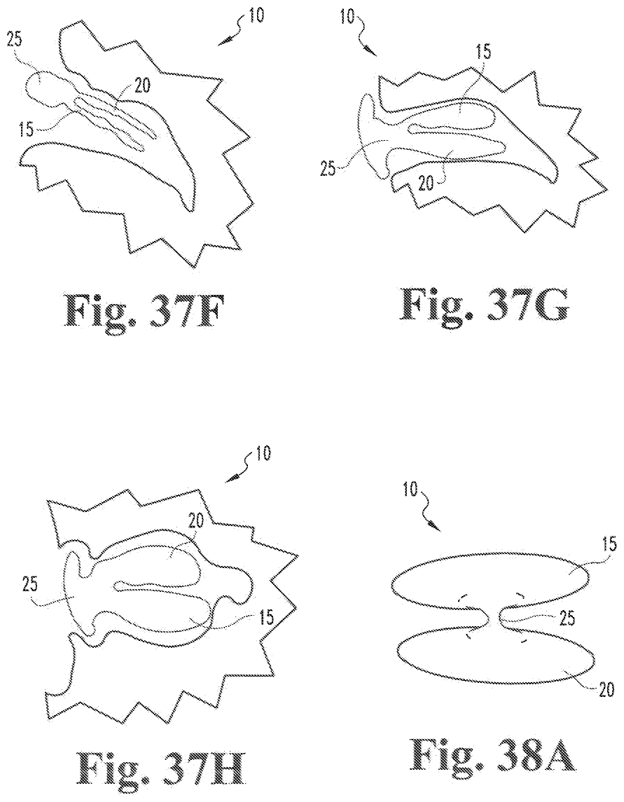

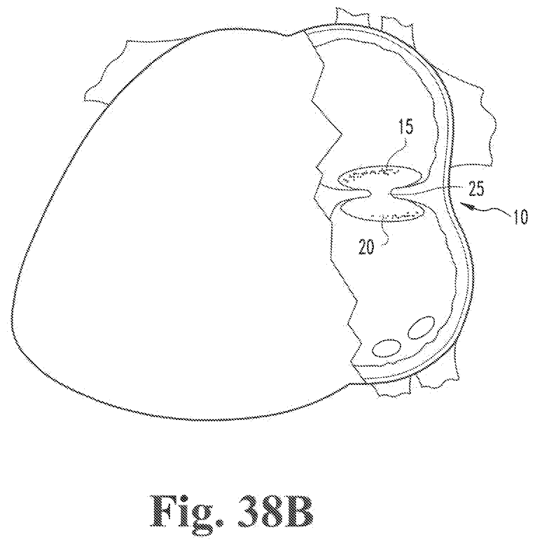

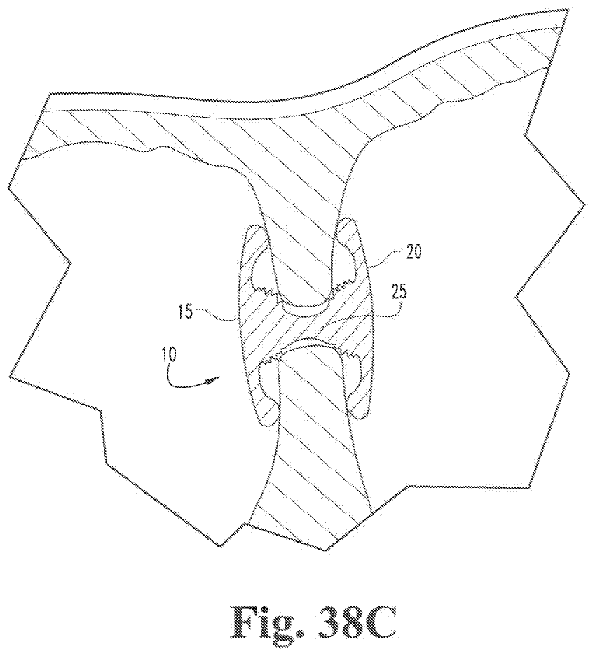

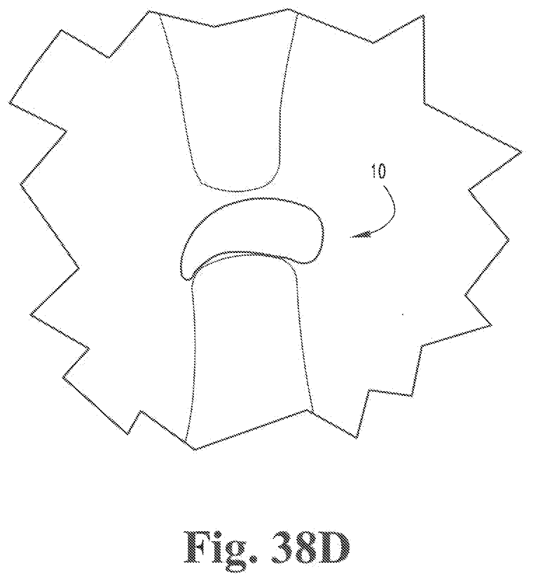

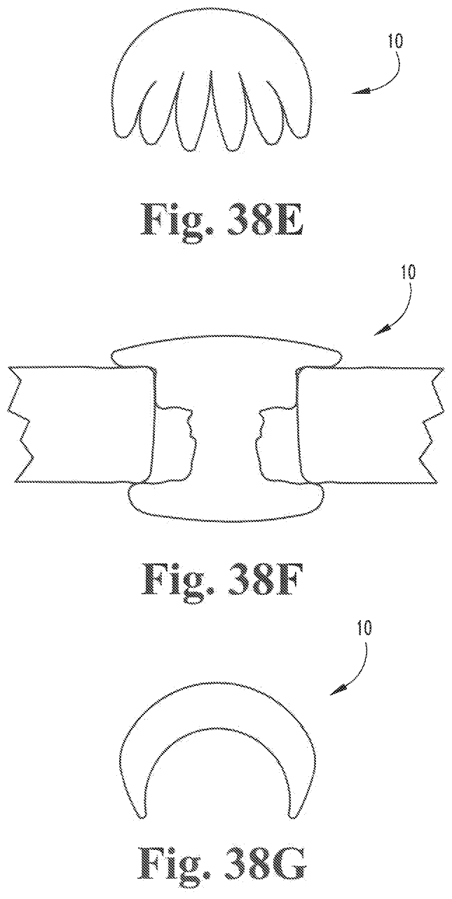

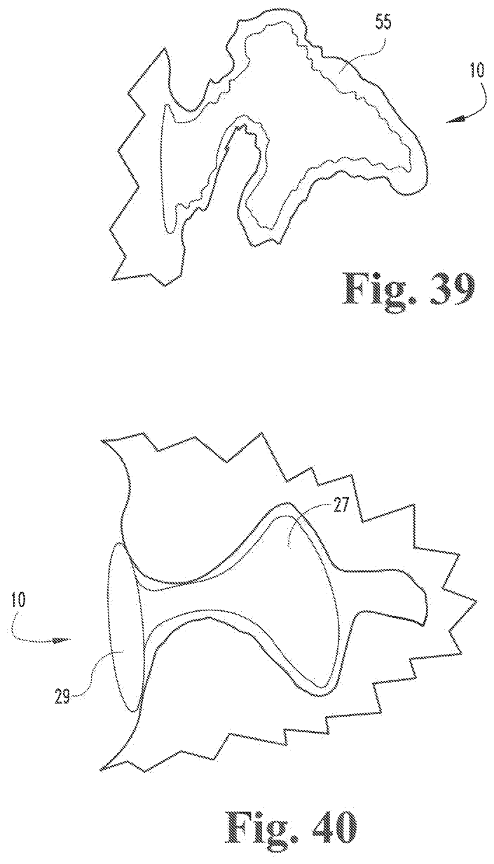

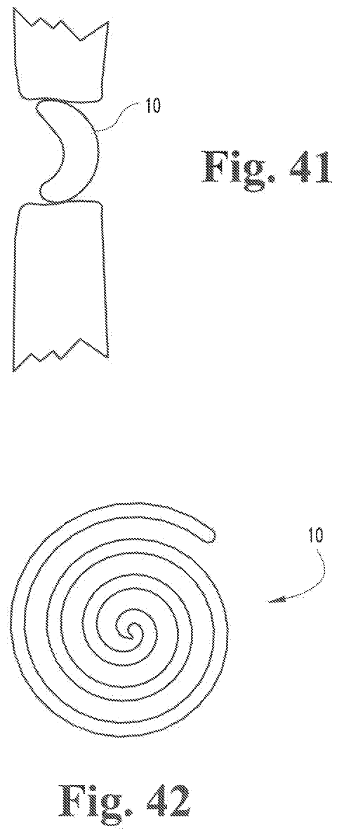

As mentioned above and illustrated in FIGS. 28-42, the device 10 is not limited to use as an atrial septal defect occluder. For example, the waist 25 may be elongated to accommodate fistulas such as urinary fistulas, gastro-intestinal (GI) fistulas, urinary GI fistulas, vaginal urinary fistulas, hepatic duct fistulas, biliary duct fistula, pulmonary fistula, and the like. Likewise, the device 10 may be curved when inflated so that it approximates or matches a perivalvular leak around a prosthetic valve. The device 10 may have one disc 15, 20 that can be deployed on one side of an abnormal communication, such as patent ductus arteriosus.

The device 10 may be made without a hub, per se, but rather having a direct connection to the filling catheter; once the device 10 is filled it is then directly sealed, as detailed above, and then disconnected from the filling catheter. The filling catheter can be advanced inside another guide catheter.

The device 10 may be made in any one of a variety of shapes when inflated, such as rectangular, oblong, star-like, cone, crescentic, curved, or the like, so as to accommodate different communications such as vascular malformation, arteriovenous (AV), and the like.

The discs 15, 20 and/or the waist 25 may be tapered when inflated for better anchoring and/or occlusion. The device 10 may consist of only one or a few tubes that may expand into a snake-like fillable chamber that expands to occluder an abnormal opening. This can be enclosed within a larger enclosure that forms the device 10.

The device 10 may consist of sequential fillable discs 15, 20 that are connected to fill larger or longer chambers such as a left atrial appendage. Each disc 15, 20 may have its own fillable channel that is connected to the hub 25. The discs 15, 20 also may expand to varying degrees for better anchoring of the device 10 based on the material in their walls and/or based on the filling pressure being applied in the respective filling channels.

The device 10 may include smaller fillable tubules within larger tubules inside each chamber. Likewise, the device 10 may consist of one or multiple, can be sequential or parallel, chambers connected to a disc-like fillable chamber. The distal chambers are used to anchor the device inside the targeted organ, such as left atrial appendage, while the disc is anchored at the opening. For example, closing off the atrial appendage from the left atrium. The device may not have to fully fill the cavity of the targeted organ, such as left atrial appendage, to achieve sealing of the targeted organ.

In some embodiments, the device 10 may be shaped so as to avoid critical structures adjacent to its desired emplacement. For example, the waist 25 may have a partial or half-circular cross-sectional shape, may be crescentic or tapered so that it does not compress adjacent structures. For example, for repair of a ventricular septal defect, the waist 25 may be shaped so that when inflated the waist 25 does not compress any adjacent cardiac conduction system. For left atrial appendage, certain parts of the device 10 may have limited expansion so that the device 10 does not compress adjacent left circumflex, cardiac veins, or the like. Part or all of the device 10 may be shaggy shaped when filled so that the device 10 matches the shape of the targeted organ, such as matching the left atrial appendage.

The device 10 may consist of multiple adjacent lobes so that it better fits multilobed organs, such as the left atrial appendage. In some embodiments, the device 10 has bulging segments that partially compress the adjacent wall for better anchoring. The device 10 may have an external disc made from metallic substance, such as nitinol, or non-metallic skeleton, that is covered with biocompatible surface, so that the distal Tillable chamber is used for anchoring while the proximal disc is used for sealing.

In some embodiments, the device 10 can have different configurations to avoid adjacent critical structures, for example the outside discs can be asymmetric or tapered design so as to not impinge on the aortic valve or tricuspid valve if the device is used in ventricular septal defects. There can be markers on the catheter or the device itself to inform the implanting doctor about the orientation of the device.

In some embodiments, the device 10 consists of only a plug that is implanted inside the abnormal communication, for example VSD or aneurysm. The plug can be curved when inflated so that it better anchors in. In other embodiments, the device 10 forms a partial loop, which may take on different shapes so as to minimize compression of adjacent structures.

The waist 25 may be smaller than the targeted opening, such as VSD itself, when inflated and may have a tapered shape or other shape so as to not compress critical structures such as the conduction system of the heart. In this case, the outside discs are used for sealing the device 10 in place.

The pressure applied within any chamber can be different than that applied in other chambers. For example, the pressure within the waist 25 may be less than within the discs 15, 20 so that it does not compress adjacent structures.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. An assembly for in vivo repair of an aperture defect, comprising:
an inflatable compliant fluid-tight exterior portion defining a left atrial portion, a spaced right atrial portion, and a waist portion connected in fluidic communication with the left atrial portion and the right atrial portion;
at least on inflatable structural member disposed within the inflatable compliant fluid-tight exterior portion;
a hub for receiving a fluid-tight connected in fluidic communication with the exterior portion;
at least one channel connected in fluidic communication with the hub and with the at least one inflatable structural member;
wherein the inflatable compliant fluid-tight exterior portion, the at least on inflatable structural member disposed within the inflatable compliant fluid-tight exterior portion, the hub, and the at least one channel connected in fluidic communication with the hub and with the at least one inflatable structural member define a device; and
wherein the device is substantially non-metallic.

2. The assembly of claim 1, wherein the at least one inflatable structural member is a plurality of inflatable structural members.

3. The assembly of claim 2, wherein the at least one channel is a plurality of respective channels, each respective channel being fluidically connected to a respective structural member.

4. The assembly of claim 1 wherein the inflatable compliant fluid-tight exterior portion is the at least one inflatable structural member.

5. The assembly of claim 1 and further comprising a first inflatable ring member positioned around the left atrial portion, wherein the first inflatable ring member is operationally connected to the left atrial portion and connected in fluidic communication with the hub.

6. The assembly of claim 6 wherein a second inflatable ring member positioned around the right atrial portion, wherein the second inflatable ring member is operationally connected to the right atrial portion and connected in fluidic communication with the hub.

7. The assembly of claim 1 wherein the inflatable compliant fluid-tight exterior portion is at least partially covered with a coating, and wherein the coating selected from the group of biocompatible materials consisting of hiruidin, fibronectin, biocompatible adhesives, standard polymers, shape memory polymer, biological tissue, polyester, nylon, polypropylene, polyethylene, fluorinated ethylene propylene, EPTFE, Polyvinyl alcohol, Dacron mesh, polyetherimide block copolymer, polyurethane, polyester fabric, biodegradable materials such as poly-L-lactide, poly-D-lactide, polyglycolide, polydioxanone, polycaprolactone, polyacetic acid, polyethylene oxide copolymers, cellulose derivatives, tyrosine-derived polycarbonates, poly-amino acids, poly-lactic co glycolide, poly-hydroxybutyrate, polyglycolic acid, polylactic acid, polyethylene glycol, collagen, polycaprolactone, hyaluronic acid, polydioxanone, adhesive proteins, co-polymers, and combinations thereof.

8. The assembly of claim 1 wherein the inflatable compliant fluid-tight exterior portion has a non-homogeneous compliance that predetermines a shape of the assembly upon inflation.

9. The assembly of claim 2 wherein each respective inflatable structural members has a predetermined shape upon inflation.

10. The assembly of claim 1 and further comprising a target disposed on the inflatable compliant fluid-tight exterior portion, wherein the target is positioned over a predetermined puncture location for deflating the assembly.

11. The assembly of claim 1 and further comprising a catheter for delivery of the device, wherein the catheter further comprises a delivery tube portion for enclosing the device; a distal end for emplacing the device; a proximal end for connecting in fluidic communication with a hydrostatic fill material source; and a pressure sensor operationally connected thereto.

12. A mechanism for in vivo repair of an atrial septal or like defect, comprising:
an occluder, further comprising:
a hub;
a waist connected in fluidic communication with the hub;
a first lobe connected in fluidic communication with the waist; and
a second lobe disposed opposite the first lobe and connected in fluidic communication with the waist;
wherein the occluder is non-metallic; and
a catheter assembly, further comprising:
a delivery tube having a proximal end and a distal end;
a pressure sensor operationally connected to the delivery tube;
a hydrostatic fill material source connected in fluidic communication with the proximal end;
wherein the distal end is adapted to connect to the hub in fluidic communication for inflating/deflating the occluder.

13. The mechanism of claim 12 and further comprising a plurality of support structures connected within at least one respective lobe and in fluidic communication with the hub.

14. The mechanism of claim 12 and further comprising a plurality of channels extending from the hub into the respective lobes and the waist.

15. The mechanism of claim 14 wherein the catheter is connectable in fluidic communication with each respective channel.

16. The mechanism of claim 12 wherein at least one respective lobe is a disc

17. The mechanism of claim 14 wherein space is left between at least two channels in a respective lobe to accommodate reentry.

18. The mechanism of claim 12 wherein the catheter assembly may deliver between 0.00001 and 1000 atmospheres of hydrostatic fill material into the occluder.

19. The mechanism of claim 18 wherein inflating the occluder device to a first pressure yields an occluder device of a first size and wherein inflating the occluder device to a second, higher pressure yields an occluder device of a second, larger size.

20. The mechanism of claim 12 wherein the waist is sufficiently inflatable to completely fill the defect.
